(19) United States
(12) Patent Application Publication
(10) Pub. No.: US 2012/0322685 A1
(43) Pub. Date: Dec. 20, 2012

(54) DEVICE FOR COLLECTING AND ANALYZING MIGRATORY TUMOR CELLS

(76) Inventors: John S. Condeelis, Bronx, NY (US); James Castracane, Albany, NY (US)

(21) Appl. No.: 13/574,831

(22) PCT Filed: Jan. 14, 2011
(86) PCT No.: PCT/US11/00078

§ 371 Date: Aug. 23, 2012

Related U.S. Application Data
(60) Provisional application No. 61/336,755, filed on Jan. 25, 2010.

(51) Int. Cl. C12Q 1/24; C40B 30/04; G01N 33/574

(52) U.S. Cl. 506/9; 435/30; 435/7.23

(57) ABSTRACT

The present invention provides a method of isolating motile cells from an animal tissue, the method comprising implanting in the animal tissue a cell trap comprising a chamber with an inlet for ingress of motile cells and a porous matrix located in the chamber comprising a chemotactic factor, for a time sufficient for the motile cells to migrate into the cell trap; removing the implanted cell trap; and retrieving the motile cells from the cell trap.

### CROSS-REFERENCE TO RELATED APPLICATION

This application claims the benefit of U.S. Provisional Patent Application No. 61/336,755, filed Jan. 25, 2010, the content of which is hereby incorporated by reference into the subject application.

### STATEMENT OF GOVERNMENT SUPPORT

This invention was made with government support under grant numbers PO1CA100324 and U54CA126511 awarded by the National Cancer Institute, National Institutes of Health, U.S. Department of Health and Human Services. The government has certain rights in the invention.

### FIELD OF THE INVENTION

The present invention relates generally to the collection and analysis of migratory cells in solid tumors for screen for metastatic potential.

### BACKGROUND OF THE INVENTION

Throughout this application various publications are referred to in brackets. Full citations for these references may be found at the end of the specification. The disclosures of these publications are hereby incorporated by reference in their entirety into the subject application to more fully describe the art to which the subject invention pertains. Additionally, the disclosure of U.S. patent application Ser. No. 11/659,514, publication no. 20080138805 is hereby incorporated by reference in its entirety.

Metastasis of adenocarcinomas involves the escape of cells from the primary tumor either via lymphatics and blood vessels, transport to and arrest in a target organ, and growth of metastases in the target organ [55]. Each of these steps is a multicomponent process, with potentially different tumor cell properties and molecules playing critical roles in different steps [55]. However, most current methods of analysis of whole tumors treat the tumor as a black box. Ideally, high resolution methods for the analysis of metastasis at the cellular level such as imaging of cells within tumors, when combined with genomic approaches, could be used to accurately evaluate the roles of specific gene products in the individual steps of metastasis at the cellular level. Similarly, as new therapies are developed, the effects of specific treatments on the individual steps in the metastatic cascade could be evaluated precisely using methods that evaluate effects at the cellular level.

The most common assays for metastatic ability in vivo have been endpoint assays. Again these assays treat the tumor as a black box. For example, in vivo injection of tumor cells (often termed “experimental metastasis”), followed by determination of the number of metastases in a target organ such as the lung is a simple method for evaluation of arrest and growth of tumor cells in target organs [56]. Detailed studies using this assay have demonstrated that extravasation per se tends not to be rate-limiting, but that growth of metastases is inefficient [57, 58]. However, this assay is limited by the introduction, in a non-physiological manner, of a bolus (typically 100,000 cells) of cells cultured in vitro. A more physiological approach to analysis of tumor cell metastasis makes use of the injection of tumor cells into a related (orthotopic) tissue, followed by growth of a primary tumor. The primary tumor then acts as the source of tumor cells for metastasis. Such “spontaneous metastasis” assays are more accurate models of human disease in that they rely upon growth of a primary tumor, and the fact that the tumor cells themselves must actively leave the primary tumor and enter the vasculature in order for metastasis to occur [59-61]. Cell lines specifically selected for high metastatic ability through use of the experimental metastasis assay are not necessarily highly metastatic in the spontaneous metastasis assay [62]. Thus, for a detailed comparison of all the various steps of metastasis, an assay such as the spontaneous metastasis assay must be utilized. However, analysis of metastasis using the spontaneous metastasis assay typically measures only the growth of the primary tumor and the number of metastases that form in a target organ. The relative efficiency of each of the steps of metastasis and the microenvironments involved can not be assessed by this type of assay.

Analysis of the individual steps in metastasis is crucial if insights at the molecular level are to be linked to the cell biology of cancer. For example, as specific cell lines are manipulated to express particular oncogenes and suppressers, the effects on metastatic ability will need to be interpreted in terms of the particular steps in the metastatic cascade that are affected at the cellular level. Likewise, the discovery of specific genes that correlate with metastatic potential cannot be related to mechanism unless the metastatic step at the cellular level in which the gene is involved is identified. A technical hurdle to achieving the analysis of the individual steps of metastasis is the fact that, at the gross level, tumors are heterogeneous in both animal models and patients. Human primary tumors show extensive variation in all properties ranging from growth and morphology of the tumor, through tumor cell density in the blood, and formation and growth of metastases. Similarly, tumor cell lines show broad variation in formation of a primary tumor and metastatic ability. For example, growth of the primary tumor is simple to quantify, and has been a useful assay for identifying genes that are important for tumor formation. However, invasive migration and entry of tumor cells into the circulation are crucial early steps in the metastatic cascade and have been assayed in various ways but only indirectly [63-65].

Human breast carcinomas in particular, the vast majority of which are ductal carcinomas, are extremely heterogeneous both clinically and pathologically. Current methods of histologic grading, such as the modified Bloom-Richardson method, allow pathologists to separate tumors into 3 strata. Distinct differences in survival rates, however, effectively apply only to those patients with very well differentiated or low grade tumors. Recurrence-free survival for grade I carcinomas at 5 and 10 years, for example, is 75% and 64% respectively, while the same figures for grade 2 carcinomas are 50% and 40%, and for grade 3 carcinomas are 40% and 38% respectively. This suggests considerable overlap in features of grade 2 and 3 tumors, with classification by grade being a very imperfect means for effective tailoring of management and prediction of outcome [66, 67]. Gene expression profiling with microarrays have shown promise in refining these groups further [68] and support the notion that the invasive and metastatic potential of the primary tumor is encoded early in the bulk of the tumor including the stroma [69].

Cancerous tumors are dynamic microenvironments that require unique analytical tools for their study. Better understanding of tumor microenvironments may reveal mechanisms behind tumor progression and generate new strategies for diagnostic marker development, which can be used routinely in histopathological analysis. It has been shown that cell invasion and intravasation are related to metastatic potential and have linked these activities to gene expression patterns seen in migratory and invasive tumor cells in vivo. Existing analytical methods for tumor microenvironments include collection of tumor cells through a catheter needle loaded with chemical or protein attractant (chemoattractant). This method has some limitations and restrictions, including time constraints of cell collection, long term anesthetization, and in vivo imaging inside the catheter. None of the existing technology predicts progression of a tumor to metastasis, allows identification of patients who would benefit from intense chemotherapy and radiation therapy, or allows personalization of therapies based on the particular patient's tumor cell profile. The present invention addresses this need.

### SUMMARY OF THE INVENTION

The present invention provides a method of isolating motile cells from an animal tissue, the method comprising implanting in the animal tissue a cell trap comprising at least one chamber with an inlet for ingress of motile cells, and a porous matrix located in the chamber comprising a chemotactic factor, for a time sufficient for the motile cells to migrate into the cell trap; removing the implanted cell trap; and retrieving the motile cells from the cell trap.

The present invention also provides a method of isolating motile cells from an animal tissue, the method comprising implanting in the animal tissue a cell trap comprising at least one chamber with an inlet for ingress of motile cells, an electrode array located in the chamber to indicate the presence of motile cells in the cell trap, a transmitter attached to the electrode array to transmit the indication of the presence of motile cells in the cell trap to an external data acquisition system, and a porous matrix located in the chamber comprising a chemotactic factor, for a time sufficient for the motile cells to migrate into the cell trap; removing the implanted cell trap; and retrieving the motile cells from the cell trap.

### BRIEF DESCRIPTION OF THE DRAWINGS

FIG. 1A-1D: Heterotransplants of human breast tumors can be prepared and invasive cells collected from them using the in vivo invasion assay. (A) Tumor surgical specimens from breast resections with different diagnoses were obtained and transplanted into the mammary fat pads of SCID mice. Best tissue growth and survival was obtained at the site shown. (B) Invasive cells were collected from the transplants using the in vivo invasion assay described. Invasiveness scored as the number of cells collected by chemotaxis to EGF per 4 hours is shown and is related to the pathological diagnosis with normal, and ductal carcinoma in situ showing only background cell invasion, while invasive lobular carcinoma and invasive ductal carcinoma show significant invasion over background. (C) In human cell line MDA-MB-231 derived mammary tumors, cells were collected in response to EGF. Both EGF and CSF1-mediated collection of invasive cells requires the activity of the EGF receptor (both are inhibited by the EGF receptor specific inhibitor Iressa) consistent with an EGF to CSF1 like paracrine loop in human cell derived tumors like that observed in rat and mouse mammary tumors. (D) Cells collected from MDA-MB-231 derived mammary tumors were typed with antibodies which demonstrated 93% tumor cells and 4% macrophages suggesting paracrine-mediated invasion.

FIG. 2: Example of a design of the cell trap of the present invention. The exploded view of the schematic shows the general principles of assembly and illustrates the operating concept. The channel with a microelectrode array spanning the bottom of the channel is presented schematically. The sponge represents the nanoporous structure infused with chemoattractant (e.g. EGF). The scored diaphragm in the glass cover plate will allow for eventual retrieval of the cells through the application of a positive pressure on the inlet side of the cell trap of the present invention. Electrical connection to the electrode array will be through the wires shown. The shape of the cell trap can be tapered as needed (not shown) to ease insertion and removal into and from tissue.

FIG. 3A-3F: Imaging techniques. (A-C) Fluorescently labeled stromal cells, when injected into mammary tumors, can be followed and their behavior and cell interactions observed in real time over several days. (A) GFP-labeled BAC macrophage (arrow) injected into a non-fluorescent PyMT mouse mammary tumor shows motility and linear extension along extracellular matrix fiber. (B) GFP-labeled BAC macrophage without functioning CSF-1 receptors (arrow) injected into a non-fluorescent PyMT tumor remains non-motile in the same tumor background. (C) GFP-labeled tumor cells (MTLn3) injected into a non-fluorescent tumor can be visualized as they interact and move on extracellular matrix fibers. (D-F) Stromal cells cell be imaged and their behavior and interactions documented in a positive (GFP-labelled) and negative (not fluorescent on a fluorescent background) format. (D) GFP-fluorescent stromal cells are seen invading into a surgically transplanted non-fluorescent human breast tumor in a C57BL/6-TgN(ACTbEGFP) transgenic mouse mammary gland. (E-F) Non-fluorescent cells are seen as shadows moving over a GFP-fluorescent background. Arrows in (E) show direction of movement. Time interval between images is 4 min. All images mag bars=25 μm. Note that (D) is at a much lower magnification.

### DETAILED DESCRIPTION OF THE INVENTION

The present invention provides a method of isolating motile cells from an animal tissue, the method comprising implanting in the animal tissue a cell trap comprising at least one chamber with an inlet for ingress of motile cells, and a porous matrix located in the chamber comprising a chemotactic factor, for a time sufficient for the motile cells to migrate into the cell trap; removing the implanted cell trap; and retrieving the motile cells from the cell trap.

The present invention also provides a method of isolating motile cells from an animal tissue, the method comprising implanting in the animal tissue a cell trap comprising at least one chamber with an inlet for ingress of motile cells, an electrode array located in the chamber to indicate the presence of motile cells in the cell trap, a transmitter attached to the electrode array to transmit the indication of the presence of motile cells in the cell trap to an external data acquisition system, and a porous matrix located in the chamber comprising a chemotactic factor, for a time sufficient for the motile cells to migrate into the cell trap; removing the implanted cell trap; and retrieving the motile cells from the cell trap.

In one embodiment, the cell trap comprises at least one chamber with an inlet for the ingress of motile cells, an electrode array located in the chamber to indicate the presence of motile cells in the cell trap, a transmitter attached to the electrode array, and a porous matrix located in the cell trap comprising a chemotactic factor. In one embodiment, the electrode array is located between the inlet and the porous matrix, and the transmitter is attached to the end of the electrode array. In another embodiment, the cell trap comprises at least one chamber with an inlet for the ingress of motile cells, and a porous matrix located in the cell trap comprising a chemotactic factor.

The cell trap is preferably made by patterning the electrode array onto a wafer substrate by any method known in the art, such as photolithography, and then placing a second wafer on top. Preferably, the substrates are bonded together. The substrates can be bonded together by any method known in the art, such as by the use of heat or of a thin film of a chemical to bond the substrates together. The wafers can be made of any biologically inert material, preferably glass or silicon. In one preferred embodiment, the substrate is Pyrex. In this preferred embodiment, one substrate is preferably pattered with the at least one chamber and the channel via photolithography and acid etching. The etched substrate is then preferably bonded together with the second substrate with a thin film of polydimethylsloxane (PDMS). [71] The porous matrix can be put into the cell trap e.g., by placing it onto the wafer substrate after patterning the electrode array and before placing the second wafer on top, or by putting the porous matrix into the cell trap after placing the second wafer on top. The finished cell trap may be packaged by any method known in the art.

The openable closure can be any known in the art, such as a membrane or an a patterned diaphragm.

The electrode array allows for the quanitification of the number of motile cells migrating into the cell trap. The electrode array is preferably designed with a plurality of interdigitating electrodes. The electrodes may be made from any material known in the art, for example, Cu/Ag or ITO. The arrival of motile cells in the cell trap changes the electrode array baseline current. This allows a quantification of the number of motile cells in the cell trap. By monitoring the electrode array's electrical signal as a function of time, the cell arrival rate and total cell number can be determined. This can be done by any means known in the art, such as by capacitative or resistive methods. Although the capacitative method may be more sensitive, the resistive approach may be more accurate. The transmitter relays the electrode array's signal from the implanted cell trap to an ex vivo data acquisition system. This can be done through a hardwired or a wireless connection. The hardwired connection could be any linkage known in the art, such as a linkage similar to a catheter guidewire. The wireless connection could be any wireless connection known in the art, such as RFID.

When the cell trap does not have an electrode array, the number of motile cells migrating into the cell trap can be measured after removal of the cell trap from the animal tissue either before or after retrieval of the cells from the cell trap by any method known in the art, for example, by retrieving the cells and counting them, or by counting the cells within the cell trap.

The cell trap may additionally comprise an etched channel running from the inlet of the cell trap towards the rear of the cell trap. The channel facilitates the movement of the motile cells towards the rear of the cell trap. The cell trap may additionally comprise an outlet located at substantially the other end of the cell trap from the inlet. Preferably, the outlet is covered by a membrane impermeable to the motile cells, allowing motile cells to enter the cell trap only from the inlet and barring motile cells within the cell trap from leaving via the outlet. Motile cells will remain inside the cell trap since motile cells have a preference to move up a chemotactic gradient. In a preferred embodiment, if the cell trap comprises more than one chamber, the chambers are located along the etched channel. In a most preferred embodiment the chambers are located sequentially along the etched channel.

The tissue can be from any animal. Preferably, the animal is a vertebrate, more preferably a mammal, for example a rodent or a human.

Any tissue from the animal can be utilized, where the tissue has motile cells that are directed toward a chemotactic factor. Additionally, any type of tissue can be used, for example tissue in culture, tissue taken from a biopsy, or tissue in a living mammal. Preferably the tissue is cancerous, a non-limiting example of which is mammary tissue.

The porous matrix can be any matrix that will allow motile cells in the tissue to move through the matrix in response to the chemotactic factor. The porous matrix may be comprised or one matrix throughout the cell trap or may be composed or one or more matrixes in separate areas of the cell trap. For example, it may be a photodefinable polymer or a biologically inert substrate that has been electrochemically etched to create a nanoporous network with high surface area to volume ratio. In preferred embodiments, the porous matrix can be a matrigel, since it is chemically similar to vertebrate extracellular matrix, or an interpenetrating matrix of miscible polymers. Preferably, an interpenetrating matrix of miscible polymers can be produced by the mixing and interpenetrating of two fluids such as PMMA and SU8 which creates a nest of randomly distributed pathways throughout the material. Once the mixture cures, the application of PMMA solvent creates nanopores within the structure of the remaining SU8 structure. In another preferred embodiment, the porous matrix is a hydrogel which is a mixture of poly-ethylene glycol diacrelate (PEGDA) and poly-ethylene glycol monoacrelate (PEGMA) which is cured. [71] The porous matrix should preferably have an extremely high surface area to volume ratio totally infiltrated by nanoscale pores which would allow the release of the chemotactic factor by self-diffusion.

The chemotactic factor can be any factor which attracts cells towards it, for example, epidermal growth factor, CSF-1, CCLx, ETs1-2, Lps, SDF-1, HGF, PDGF B/B, FGF-1, VEGF-α, Heregulin, TGF-α, or fetal serum. Preferably, the chemotactic factor will attract cancer cells. Most preferably, motile cancer cells will move up the gradient of the chemotactic factor and not down the chemotactic factor gradient. When the motile cells of interest are cancer cells, epidermal growth factor is the preferred chemotactic factor. When the motile cells of interest are macrophages, CSF-1 is the preferred chemotactic factor.

The cell trap can be implanted by any means known in the art, such as by surgery. The period of implantation which is sufficient for the motile cells of interest to migrate into the porous matrix can be between two hours and a few weeks. Preferably, the period of implantation is to be longer than two hours.

The cell trap can be removed by any method known in the art, such as surgery. In order to aid in removal, the cell trap may have a small length of wire or small lip on the cell trap with would allow the mechanical removal of the cell trap without damaging the cell trap.

After removal of the cell trap, the motile cells can be retrieved by any method known in the art, such as by digesting the cell attachments to the porous matrix. This can be achieved by, for example, flowing a trypsin containing buffer through the cell trap. The motile cells can then be expelled from the cell trap by any method known in the art. In a preferred embodiment, the cell trap contains an outlet covered by a membrane which can be burst, for example, by the application of positive pressure through the cell trap, allowing the expulsion of the motile cells from the cell trap. Alternatively, the porous matrix with the motile cells can be expelled from the cell trap and the motile cells can then be separated from the porous membrane by any method known in the art. In a preferred embodiment, the cell trap contains an outlet covered by a membrane which can be burst, for example, by the application of positive pressure through the cell trap, allowing the expulsion of the porous matrix with the motile cells from the cell trap.

Once the motile cells have been retrieved from the cell trap, motile cells of interest can be separated from the other motile cells. This can be done by any means known in the art such as, for example, combining the motile cells with microbeads with a binding partner to a surface marker present on the other motile cells but not the motile cells of interest. Removing the microbeads with the bound other motile cells leaves only the motile cells of interest. In another embodiment, the motile cells of interest can be separated from the other motile cells by combining the motile cells with microbeads with a binding partner to a surface marker present on the motile cells of interest but not the other motile cells. The motile cells of interest bind to the microbeads and can be removed. Preferably, the motile cells of interest can then be removed from the microbeads by any method known in the art.

Any binding partner capable of binding to the other motile cells but not the motile cells of interest, and capable of being bound (covalently or noncovalently) to a microbead can be used. For example, such binding partners can include aptamers, or preferably antibodies or antibody fragments, where the binding site is preferably specific for a cell surface marker present on the surface of the other motile cells but not the motile cells of interest. For example, when the motile cells of interest are carcinoma cells and the other cells are macrophages, a preferred microbead has antibodies specific for CD11b. The skilled artisan could formulate a binding partner for any particular motile cell/motile cell of interest combination without undue experimentation.

Aptamers are single stranded oligonucleotides or oligonucleotide analogs that bind to a particular target molecule, such as a protein or small molecule. Thus, aptamers are the oligonucleotide analogy to antibodies. However, aptamers are smaller than antibodies and aptamer binding is highly dependent on the secondary structure formed by the aptamer oligonucleotide. Both RNA and single-stranded DNA, or DNA analog, aptamers are known. Aptamers that bind to virtually any particular target can be selected by using an iterative process called SELEX (Systematic Evolution of Ligands by EXponential enrichment).

An antibody or antibody fragments can be polyclonal, monoclonal, or recombinant and can be of any animal, such as a rodent or a human, or a mixture of animals, such as humanized mouse.

Any type of microbead can be used to bind the other motile cells. For example, the microbeads can be heavy particles that are pelleted under centrifugal conditions, but which do not pellet the motile cells of interest. Alternatively, the microbeads can be buoyant particles that are not pelleted under centrifugal conditions that pellet the motile cells of interest. In a preferred embodiment, the microbeads are magnetic beads.

The motile cells of interest may be cancer cells such as cancer stem cells or cells from any type of cancer, such as a carcinoma, but the motile cells of interest are not limited to cancer cells, and can be normal stromal cells such as macrophages. When the motile cells of interest are cancer cells, the other motile cells may be macrophages or other normal stromal cells, such as fibroblasts or eosinophils. The predominant type of other motile cell may vary depending on the type of tissue the motile cells are taken from and the identity of the motile cell of interest. A skilled artisan could formulate a binding partner for the effective removal of each type of other motile cell without undue experimentation. The other motile cells, which are isolated on the microbeads, can be retained for further analysis. Such further analysis may include quantification of the cells, or analysis of mRNA or protein expression.

Removing the microbeads bound to the other motile cells isolates the motile cells of interest so that further analysis or cell culture can be performed. For example, the motile cells of interest can be quantified, in order to approximate the number of motile cells of interest in a given amount of tissue, in order to compare the number of motile cells of interest to the amount of the other motile cells. The motile cells of interest can be quantified by any method known in the art, such as by microscopic observation.

In some preferred embodiments, mRNA or protein expression of at least one gene is determined in the motile cells of interest. In some preferred embodiments, the mRNA or protein expression of the motile cells of interest may then be compared to the expression of the same gene or genes in nonmotile cancer cell or noncancerous cell from the same tissue. When analysis of mRNA or protein expression of more than one gene is desired, microarray technology can be employed. This well-established technology can analyze mRNA or protein expressions of many genes at once, allowing comparison of, for example, an entire genome between motile cells of interest and nonmotile cancer cells from the same tissue. Generally only a few hundred motile cells of interest will be isolated, typically providing only 20-50 ng of total RNA, which may be insufficient for analysis. In such a case, mRNA from the motile cells of interest can be amplified prior to determination of gene expression. Such amplification can be done by any method known in the art, such as by reverse transcription and cDNA amplification

### EXPERIMENTAL DETAILS

### Development

### Animal Models Used to Define Invasive Cancer Cells in Mammary Tumor

Two types of animal models were established for the intravital imaging of the migratory population of carcinoma cells and stromal cells within the primary tumor. (1) Immunocompetent Fisher rats with rat mammary carcinoma cell lines of various metastatic potential injected orthotopically into a mammary fat pad. All cell lines stably express green fluorescent protein (GFP) from constitutive promoters making the carcinoma cells of the resulting primary tumor visible for imaging at cellular resolution [2-5]. (2) Transgenic mice that express GFP from tissue specific promoters crossed with mice that express oncogenes from the MMTV promoter generating tumors with different fluorescent cell types thereby making these cells in the primary tumors of the mammary gland visible for imaging at cellular resolution [1, 6-10, 12].

The transgenic mouse models created are: (a) MMTV-PyMT×WAP-Cre/CAG-CAT-EGFP or MMTV-Cre/CAG-CAT-EGFP mice which are mice with GFP-fluorescent carcinoma cells in PyMT oncogene generated tumors. These mammary tumors were used to image migratory cells and their accompanying stromal cells. Stromal cells were imaged as “shadows” since they scattered light from the fluorescent cells in the tumor [1, 2, 4]. (b) To visualize tumor cells and macrophages simultaneously in the same animal, a mouse model was prepared by breeding mice with genetic susceptibility to mammary tumors expressing GFP with mice expressing CFP in their myelomonocytic cells=MMTV-PyMT×WAP-Cre/CAG-CAT-EGFP or MMTV-Cre/CAG-CAT-EGFP×lys-CFPKi. (c) Tie2/GFP transgenic mice were obtained from Dr. Richard Lang, (Skirball Institute, NYU, NY). Tie2 is expressed specifically in endothelial cells and consequently, GFP can mark the vasculature at sites of Tie2 expression [20] in PyMT generated tumors. Thus the following cross was prepared: Tie2-GFP×MMTV-PyMT.

Combining the animal models with multiphoton imaging the behavior of the migratory population of carcinoma cells and stromal cells during invasion and intravasation within primary mammary tumors of both rats and mice were able to be studied. The summary of the results obtained with both rats and mice as published in references 1-6, and 9-10 is as follows:

(a) Carcinoma cells in the primary tumour can move at up to 10 times the velocity of similar cells in vitro.
(b) The highest velocities are observed for carcinoma cells in metastatic tumours that are moving along linear paths in association with extracellular-matrix (ECM) fibers.
(c) The ECM and its interaction with carcinoma cells can be observed directly using the second harmonic signal from multiphoton-illuminated tumors.
(d) Carcinoma cell motility is characterized as solitary amoeboid movement and is unrestricted by networks of ECM in mammary tumors except around blood vessels.
(e) Carcinoma cell motility is restricted at the basement membrane of blood vessels, where the cells must squeeze through small pores in the basement membrane/endothelium to gain access to the blood space.
(f) Carcinoma cells in non-metastatic tumors are fragmented during intravasation as they squeeze across the basement membrane/endothelium, whereas carcinoma cells in metastatic tumours cross this restriction as intact cells.
(g) Carcinoma cells in metastatic tumors are attracted to blood vessels, where they form a layer of cells that are morphologically polarized towards the vessel.
(h) Chemotaxis to epidermal growth factor is shown by carcinoma cells in vitro and in vivo in primary tumors, and might be responsible for the attraction of carcinoma cells to blood vessels.
(i) Polarization of tumor cells toward blood vessels is correlated with increased intravasation and metastasis.
(j) Metastatic mammary tumors contain large numbers of rapidly moving macrophages and other leukocytes near blood vessels. They are a source of chemotactic cytokines.
(k) Intravital imaging can be productively correlated with gene-expression profiling to generate new insights into the expression patterns that are responsible for invasive-cell behavior.

### Invasion and Intravasation Involves Chemotaxis In Vivo

Items g-j, above, suggest that chemotaxis toward blood vessels is an important step in intravasation (involving EGF receptor ligands), angiogenesis, and infiltration of tumors by immune cells [16]. Therefore, an in vivo invasion assay was created in which three needles (microneedles ˜100 um in diameter) containing matrigel and various growth factors are placed independently with microscopic precision, using a specially designed micromanipulator guided device, into the primary tumor for chemotaxis-based cell collection in vivo [3]. One needle contains control conditions (usually no ligand or an inhibitor), and the other two are identical [3]. Multiphoton-based intravital imaging was also used to document the behavior of cells around the needles and demonstrated that it was similar to their behavior around blood vessels [6, 8-9, 14]. These methods demonstrate that EGF receptor ligands are sufficient to cause chemotaxis to the indwelling needles while many other growth factors failed. Fetal serum was also sufficient but this required signaling by the EGF receptor again implicating the EGF receptor. Furthermore, CSF-1 was also sufficient for cell collection. Gradients of either EGF or CSF-1 stimulated collection into needles of both tumor cells and macrophages even though tumor cells express only EGF receptor and macrophages only CSF-1 receptor. Intravital imaging demonstrated that macrophages and tumor cells chemotax toward needles containing either EGF or CSF-1 [9, 12]. Migration and collection was directly correlated with macrophage density in the mammary gland, stage of progression to malignancy (only background levels of cells, <200 cells, were collected from normal mammary glands and adenoma stage tumors compared with carcinoma stage tumors, ˜1000 cells) and the presence of CSF1 in the tumor was required for cell collection as shown using mouse models with either decreased (op/op) or increased (tet on CSF-1) CSF-1 expression that regulates macrophage recruitment to the mammary gland and metastasis [9]. Inhibition of either CSF-1- or EGF-stimulated signaling using receptor directed inhibitors blocked the migration and needle collection of both cell types [9], and intravasation. Furthermore, the motility of tumor cells and intravasation occurs preferentially in association with macrophages [9, 11, 14]. This work provides the first direct evidence for a paracrine interaction between macrophages and tumor cells during cell migration in vivo and indicates a mechanism for how macrophages might contribute to metastasis [9, 15].

### Development of Techniques to Profile and Compare Chemotactic Tumor Cells

cDNA microarrays have the potential to identify the genes involved in invasion and metastasis. However, when used with whole tumor tissue, the results average the expression patterns of different cell types. The in vivo invasion assay, which uses chemotaxis-based cell collection of the migratory subpopulation of cells within the primary tumor, was combined with array-based gene expression analysis to identify the gene expression patterns correlated with carcinoma cell chemotaxis and invasion. Cell collection from mammary tumors of rats and mice using this method shows that carcinoma cells and macrophages constitute the invasive cell population [9].

### Extending the Approach to Human Tumors

Many human tumors can be grown in immunodeficient SCID and Rag1 mice. The source of human tumor tissue is surgical resections. Fresh non-necrotic tumor samples of 1-2 mm³ are inoculated subcutaneously into the mice. These are called heterotransplants and have several advantages [21]. (1) The tumor and stroma are from the same individual and, thus, histocompatibility differences are not a concern. (2) Many of the features of the natural tumor stroma will be present within the microenvironment of the heterotransplant including the tumor extracellular matrix, cells of the vasculature and tumor specific leukocytes. If human tumors are transplanted into SCID mice with minimal in vitro manipulation, the original histology of the tumor can be maintained in the xenograft for up 4 (some reports of up to 22) weeks [22]. Heterotransplantation has been done successfully with both SCID and Rag1 mice [21, 23]. Each has advantages with SCID being better characterized for this purpose and Rag1 showing less B and T cell leakiness. Heterotransplantation has been studied previously in many tumors including leukemia [24], renal cell carcinoma [25], breast carcinoma [26, 27], medulloblastoma [28], head and neck cancer [29], colorectal cancer [30], ovarian carcinoma [31], osteosarcoma [32], and lung cancer [33]. Studies with human lung cancers (non-small cell lung cancer tumors resected from patients) demonstrate a high take (47%) on initial transplantation, histopathology identical to the original tumor at least as far as the first mouse to mouse transplantation, identical sensitivity to pacitaxel as observed in clinical studies [34], and overall gene expression profile similarity to that observed in the original tumor [21]. These results predict that these heterotransplants are suitable models for the study of metastasis in human tumors and for the identification of cell behavior and gene expression profiles associated with the migratory and invasive cells of human tumors using the methods described for rat and mouse tumors.

Human lung tumors were heterotransplanted into nude mice as described [34], and tumors from the first mouse to mouse transplant were used in an in vivo invasion assay to collect cells using chemotaxis to needles. From the lung tumors, 2.5 fold more cells were collected in needles containing 25 nM EGF and 2 fold more in needles containing 50 nM Heregulin as compared to needles with buffer alone. Cell typing demonstrated that the migratory cells were 73% carcinoma, 17% fibroblasts and 8% macrophages indicating differences between the stromal cells involved in invasion of lung and mammary tumors since no fibroblasts were detected in invasive cells from mammary tumors.

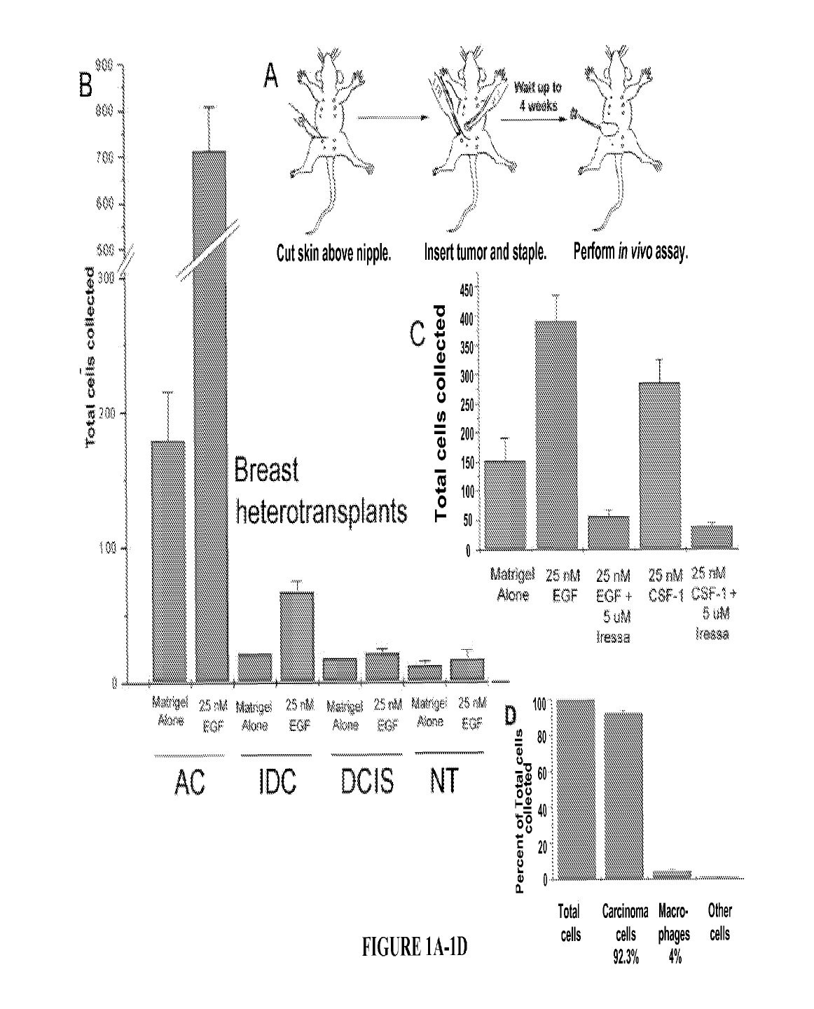

1-2 mm³ tumor surgical specimens from breast resections with different diagnoses have been obtained and transplanted into the mammary fat pads of SCID mice as shown in FIG. 1A. Best tissue growth and survival was obtained at the mammary gland site shown. Invasive cells were collected from the transplants beginning 14 days after surgery using the in vivo invasion assay. Invasiveness scored as the number of cells collected by chemotaxis to EGF per 4 hours is shown in FIG. 1B and follows the pathological diagnosis with normal, and ductal carcinoma in situ showing only background cell invasion, while invasive lobular carcinoma and invasive ductal carcinoma show significant invasion over background.

Mammary tumors derived by injecting the human cell line MDA-MB-231 into a mammary fat pad of mice were used in the in vivo invasion assay. Here, 2.5 fold more cells were collected in response to EGF (FIG. 1C) and, upon cell typing, were shown to be 93% carcinoma cells and 4% macrophages (FIG. 1D). The involvement of EGF and CSF1 in carcinoma cell invasion has been analyzed from primary breast tumors derived from the injection of MDA-MB-231 cells into the mammary fat pads of SCID mice. Both EGF and CSF1-mediated collection of invasive cells requires the activity of the EGF receptor (that is, both tumor cell and macrophage invasion is inhibited by the EGF receptor specific inhibitor Iressa) consistent with the involvement of EGF to CSF1 like paracrine loop in human cell derived tumors like that observed in rat and mouse mammary tumors. Reconstitution of invasion by MDA-MB-231 cells using the in vitro invasion assay indicates that MDA-MB-231 cells, unlike rat and mouse mammary tumor cells, are capable of invading in the absence of macrophages but only after exposure to macrophages overnight.

### Nano-Components

Data has been gathered on the main components of the design and include the wafer-level fabrication of electrode arrays, nanoscale treatment of surfaces and fluidic networks [70]. The process flow is a wafer-scale approach and thus, retains the economies of scale offered by this method in the simultaneous mass production of a wide variety of designs and exploitation of existing fabrication and metrology tools available.

The fabrication and use of arrays for electrical monitoring of cell dynamics is augmented by the incorporation of nanofluidics to complete the integrated system. Custom arrays of fluidic channels can be used for bionanotechnology applications. For example, a photodefinable polymer (SU8) can be used to create a network of covered channels for transport of biofluids through a chip. In various fabrication experiments, channel dimensions ranging from sub-micron to hundreds of microns have been successfully produced. Further, the inner surfaces have been functionalized to address specific aspects (e.g. hydrophobic/philic) of cell dynamics through the application of nanoscale coatings. A variety of flow control components, active and passive, have been produced to coordinate the mixing, transport and distribution of biofluids, reagents and gasses. An active microvalve can be used to control the flow of fluids in these systems. In this case, the 30 micron valve is constructed from polysilicon and the gap is electrostatically actuated for flow control. Using these capabilities, the incorporation and control of cells and culture medium was successfully demonstrated. Fluorescently tagged FT cells were suspended in culture medium and, under a pressure, flowed downstream. This verified that using this material (SU8) as a fluidic structural material, wall adhesion is not an issue thus validating its inclusion in the prototype.

Finally, the creation and implementation of nanoporous structures in a variety of substrate materials has been established [19]. For example, a porous network can be created in Si using electrochemical etching methods using a combination of IR illumination, solvents and impressed potential on the substrate materials to create a network of pathways ranging in size from 100 nm to 2 microns. Depending on the exact details of the fabrication method, these pathways can either be direct tunnels through the material or a circuitous maze to create a large surface area to volume architecture. This nanoporous network can then be infused with a variety of materials depending on application.

### Design and Performance

Human Heterotransplant and Human Cell Line-Derived Breast Tumors for Use with the Implantable Cell Trap

Tumor surgical specimens of human breast tumors will be obtained on a regular basis and transplanted into GFP-expressing immunosuppressed SCID and Rag1 mice and allowed to develop 3 weeks and then used within 4 weeks of implantation. It has been determined that 20% of the 1-2 mm³ pieces of breast tumors grow as heterotransplants in mice on first implantation in the mammary gland as expected [27]. This has allowed all heterotransplant experiments to be performed at orthotopic sites.

### Fabrication of the Implantable Cell Trap

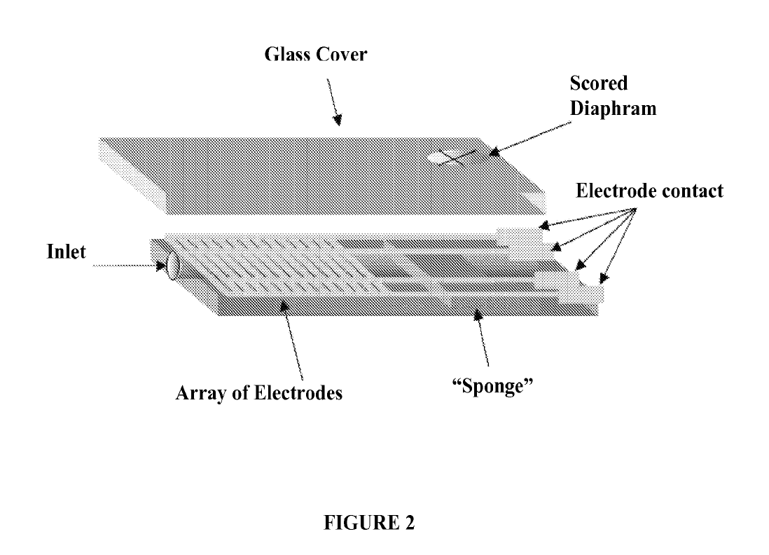

The general design of the implantable cell trap consists of a multi-component fluidic “cell trap” incorporating a method for cell attraction, capture, quantification and reporting. The basic structure of the implantable cell trap consists of a channel conduit fabricated using IC production methods and integrating the chemoattractant, cellular adhesive (ie. extracellular matrix), electrode array and fluidic components for cell retrieval. Prototypes of the implantable cell trap are fashioned from a variety of materials to test efficacy of production methods. One potential path will be to leverage experience with photodefinable polymers to create the implantable cell traps with dimensions ranging from 50-100 microns in width, 20-50 microns in height and 3-5 mm in length. This volume will allow for the capture of approximately 10,000-20,000 cells. FIG. 2 shows a top view of an example of the implantable cell trap. As can be seen, the cells enter from the left end of the channel opening and migrate toward the electrode array. This movement is stimulated by the gradient of the EGF from the nanoporous “sponge”. The electrode array is connected to an ex vivo data acquisition system through the contacts on the right side of the device to register the change in electrical characteristics of the system caused by the entry of the cells. The scored diaphragm on the topside of the right end of the channel is used to remove the cells once the implantable cell trap has been withdrawn.

Specifically, the initial proposed process begins with the patterning of the electrode array on the wafer substrate of glass, silicon, etc. For biocompatibility reasons, the initial substrate chosen is a glass wafer. The electrode array consists of a set of interdigitated fingers to allow for electrical contact with the captured cells. Extending the capabilities of the preliminary work cited above, a feature size reduction will be attempted to scale down the exposed contact area yet retaining adequate surface for the electrical measurements. It is anticipated that a series of electrode sizes ranging from 10 microns to 500 nm can be patterned for testing purposes. Adhesion, conductivity and mechanical stability will be verified to reach a final determination of array configuration. Measurement of cell arrival rate and total accumulation will be quantified through a change in the electrical performance of the circuit consisting of the cell, electrode array, leads to contact bond pads, and external measurement circuitry. Many methods are possible for efficiently cataloging this signal. The exact transduction method can be consist of either a capacitive or resistive measurement. This decision will be based on expected signal strength, exact cell dynamics and subsequent experiments. Once patterned, the array can be overcoated with the application of a polymer structural material to create the channel shape. Exploiting wafer-level production methods, a wide variety of configurations can be fabricated simultaneously.

The next step in the process is to photolithographically open selected portions of the array in the base of the channel to allow eventual contact with trapped cells. At this point, the creation of the “bait” will take place. Using production methods associated with fabrication of nanoporous structures, a “sponge-like” feature can be created at one end of the channel. This technique allows for wide selectivity in both material and pore size. Direct extension of existing methods can be used to create this feature of the implantable cell trap. However, one novel approach to create the nanopores will be to produce an interpenetrating network (IPN) of miscible polymers such as PMMA and SU8. The mixing and interpenetration of the two fluids will create a nest of randomly distributed pathways throughout the material. This mixture can be applied and patterned thus making the process step compatible with the proposed approach. Once this mixture has cured, the application of the PMMA solvent creates the nanopores within the structure leaving behind the SU8 structure. Experiments have been performed to determine the optimum mixing ratio, efficient application method to confine the IPN to the chosen section of the implantable cell trap and solvent effects on the structural components of the device. The goal is to produce an extremely high surface area to volume ratio construct totally infiltrated by nanoscale pores. The optimized “sponge” has been shown to release the EGF by self-diffusion down the channel of the implantable cell trap to attract the cancer cells toward it as in the existing catheter-based in vivo invasion assay and, by default, across the electrode array where they adhere and be counted and reported.

Returning to the details of the process flow, one of the key features of the implantable cell trap is to allow the cells to adhere to the surface of the electrode array. Previous results with the in vivo invasion assay have demonstrated that tumor cells, when confronted with a gradient of EGF on an adherent surface, do not exhibit backward migration. This results in a captive cell population. The use of various custom cell adhesion substances (extracellular matrix) which have been studied in this regard previously [11], including collagen and matrigel, will be explored to arrive at an optimum choice. There is extensive experience on suitable combinations of binding molecules, either naturally occurring or artificially fabricated at the molecular level to accomplish this task. Nanoengineering of selected combinations of macromolecules and integration with extracellular matrices such as Matrigel, collagen type A, fibronectin, etc. will be coupled to functionalized conducting polymers to complete the “trap”. The down selection will be based on several factors (in addition to adhesion efficiency) including ease of integration and electrical properties. These electrical properties will, in part, determine the sensing method. While capacitive methods may be more sensitive, if the adhesive is conductive, then a resistive approach will be attempted. Independent of the final choice, the basic mode of measurement will be that the change in the electrical signal will be monitored as a function of time to quantify cell arrival rate and integrated to give total number of cells. The electrical signals is relayed to the ex vivo data acquisition system through a hardwired connection. This connection will serve two purposes: signal relay and mechanical retraction. The electrical contacts will consist of fine gauge wires connected to the output bond pads of the cell trap of the present invention and will be encapsulated in the polymer to ensure electrical isolation. More sophisticated approaches for signal transmission can be envisioned in subsequent generations of the cell trap including RF broadcast thus breaking the physical connection to the device and allowing for long term implantation. For mechanical retraction, the initial approach will be to simply incorporate a linkage similar to the guide wire used in catheter-based instruments.

Following successful attraction, capture and recording of the cell dynamics, the remaining piece of the performance requirements of the implantable cell trap is to withdraw the device from the tumor and retrieve the cell population. Retrieved cells will be typed and RNA extracted for expression analysis as described below. Retrieval can be accomplished by flowing a trypsin containing buffer to digest cell attachments to the extracellular matrix inside the catheter. The inclusion of an exit orifice at the proximal end of the implantable cell trap is created using another patterning and processing sequence on a second glass wafer. This pattern will be fabricated using a suitable mask which will align this top wafer to the implantable cell trap bottom wafer. A thin (˜50 microns), circular (diameter: ˜50 microns) membrane will be etched in this glass wafer at the correct spot on the SU8 channel. The inclusion of the diaphragm serves two purposes: it effectively seals the proximal end of the implantable cell trap channel and thus, limits cell entry only to the distal end and secondly, it can be opened for cell flushing using positive pressure. An I/O port can be interfaced to the open end of the implantable cell trap and connected to a reservoir of fluid using e.g., a syringe. This passive diaphragm approach simplifies the design and fabrication. No external power is required to control an active valve and additional operational issues such as flow blockage or valve failure need not be considered. To control the operation of this diaphragm, the membrane will be cross-scored using etching methods to adjust the required burst pressure and to limit fragmentation of the membrane during the process. Once these fabrication steps have been completed, the two processed glass wafers are joined through the use of wafer-level bonding methods. At this point, the compound wafer stack is diced and electrical connections made to the electrode array. The small size scale of the cell traps and the large wafer format to be used for fabrication ensures a large number of prototypes can be produced and performance statistics can be quickly acquired. This results in rapid development progress and timely feedback for improvements in the device design.

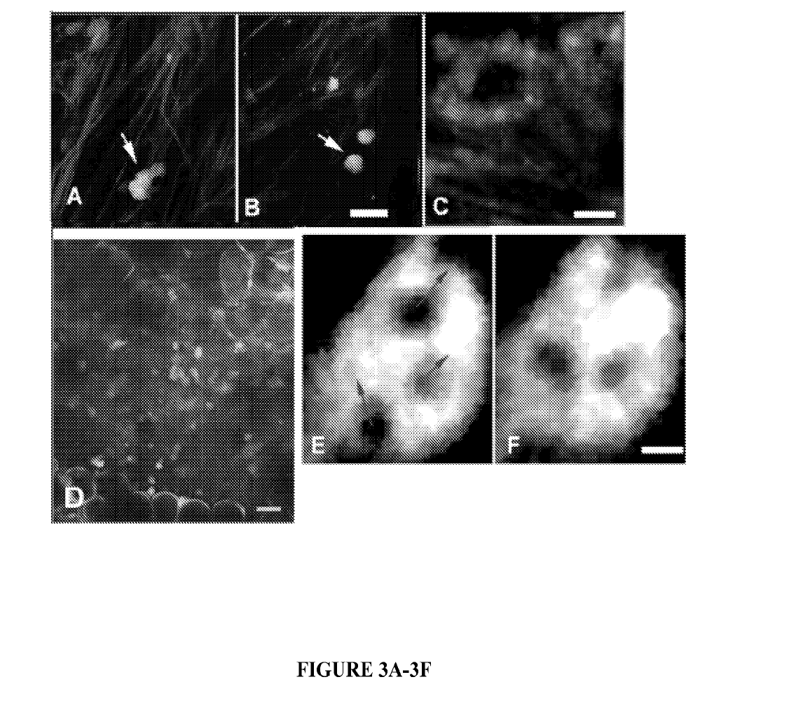

Active cell collection has been observed and validated using intravital multiphoton imaging with the existing generation of devices. The value of intravital imaging and the in vivo invasion assay resulting in needle collection of invasive cells, in classifying rat and mouse tumors as metastatic or nonmetastatic, and in characterizing the behaviors and properties of invasive tumor and stromal cells in rat and mouse tumors, is well established and documented. Intravital multiphoton imaging will be repeated initially on the well characterized rat and mouse tumors described such that the behavior of tumor cells around catheters containing various dendromer designs and growth factors can be documented (Table 1). This will allow documentation of the efficiency of cell collection and chemotaxis without withdrawing the catheter and demonstrate that cell collection is an active process. In these animal models, all tumor cells are GFP expressing allowing direct detection of individual tumor cells and indirect detection of accompanying host cells as described. (FIG. 3)

TABLE 1
Factors for use in the in vivo invasion assay (Not an exhaustive list. Good inhibitors are available for all listed.)

| Growth factor | Rationale for use |
|---|---|
| Fetal serum | Starting material which contains a broad spectrum of invasion factors. In rat and mouse mammary tumors, cells |
| | are attracted to blood vessels where they intravasate [3, 8-9]. |
| EGF | EGF receptor expression is correlated with poor prognosis in breast cancer. EGF and TGFa are chemotactic for |
| | breast carcinoma cells in vivo [9, 11, 38-40] |
| TGF-α | See above |
| CSF-1 | CSF-1 and CSF-1-receptor expression is correlated with invasive mammary tumors in both human populations and |
| | animal models. CSF-1 is chemotactic for macrophages in vivo. [38, 41] |
| Heregulin | Heregulin has been shown to enhance motility and migration of cancer cells and as the ligand for the ErbB2/ErbB3 |
| | heterodimer has been shown to enhance cell proliferation in breast cancers. [42] |
| VEGF-a | VEGF-α is correlated with angiogenic response and has been shown to stimulate invasion in breast cancer cells. |
| | [43, 44] |
| FGF-1 | FGF-1 expression is correlated with malignancy of breast tumors. [45] |
| PDGF B/B | PDGF B/B is produced by macrophages and stimulates cell motility in connective tissue cells, monocytes and |
| | neutrophils, and is correlated with invasion in a number of human cancers. Furthermore, PDGF receptor beta, |
| | which responds to PDGF B/B is found on monocytes and macrophages. [46-49] |
| HGF | HGF stimulates tumor cell-cell interactions, matrix adhesion, migration, invasion, and angiogenesis. [50] |
| SDF-1 | The chemokine SDF-1 induces chemotaxis and invasion in breast cancer cells. [51-52] |
| Lpa | LPA stimulates cell proliferation, migration and survival by acting on its cognate G-protein-coupled receptors. |
| | [53] |
| ETs1-3 | Endothelins 1-3 and their receptors are reported to mediate chemotaxis in a variety of cell types including |
| | carcinoma cells. It is proposed that ETs are involved in invasion of breast tumors. [54] |
| CCLx | The CC chemokines CCL2, CCL3, CCL4, CCL5, and CCL8 are involved in leukocyte recruitment in mammary |
| | tumors [52]. |

### Determining Gene Expression Profiles of Invasive Cells Collected Using the Implantable Cell Trap of the Present Invention

To use the cDNA and oligonucleotide microarray on invasive tumor cells it is necessary to collect migratory tumor cells with the implantable cell trap, retrieve the cells from the capsule and then separate the different cell types collected into pure populations. In previous work it was shown that tumor cells and macrophages invade together in breast tumors and that they can be separated using antibodies directed against cell type specific cell surface antigens on magnetic beads [7, 11-12]. Collection using the implantable cell trap should have the same involvement of macrophages in tumor invasion as with the in vivo invasion assay since invasion and metastasis in PyMT tumors follows progression to malignancy that is similar to that seen in human breast tumors and appears to be a general property of breast tumors which should be repeated in collection by the implantable cell trap of the present invention if it measures invasion faithfully [36]. Furthermore, the results described in FIG. 1 with tumors derived from the human breast carcinoma cell line MDA-MB-231 demonstrate that macrophages and tumor cells invade together as in PyMT tumors.

Once a pure population of invasive tumor cells is obtained, expression analysis using the amplification and microarray methods described herein and in supporting publications [5, 7, 11-12] will be used to identify genes regulated selectively in the cell trap of the present invention collected migratory and invasive tumor cell population for each primary tumor. The expression patterns of invasive cells collected into the implantable cell trap will be compared to tumor cells obtained from the whole primary tumor. Tumor cells from the whole tumor (resident) will be purified by mechanical disruption and isolation of tumor cells on beads with antibodies directed against cell type specific cell surface markers [7, 12, 17, 37]. Invasive tumor cells will also be purified from macrophages and/or other cell types using antibody beads directed against cell surface antigens such as Cd11b [7]. The resident and invasive tumor cell expression profiles will be determined on oligo and cDNA microarrays using the amplification and analysis methods described previously [5, 7, 12-13]. Controls will include those used for the data shown in this application: changes in gene expression of tumor cells in response to exposure to matrigel, growth factor used for cell collection, binding of antibody and bead isolation, and holding cells within the collection needle within the tumor. The effects of each treatment will be determined by comparing expression patterns of cells exposed to each treatment to expression patterns of cells that have not. Changes in gene expression resulting from these manipulations will be determined relative to those that only result from the primary tumor environment. To date, the expression patterns of invasive tumor cells from both rat and mouse mammary tumors have been documented to result from the environment of the primary tumor and cannot be produced by any of the manipulations described in these controls that mimic the methods used to isolate cells. Finally, the amplification of RNA from invasive cells will be characterized and only those preps that can be validated for fidelity of amplification as described previously [7] will be used.

### Discussion

Using multiphoton imaging it has been discovered that metastatic tumor cells are chemotactic to blood vessels. By following the behavior of tumor cells around blood vessels, criteria were derived for the construction of a device that acts as an artificial blood vessel. That is, metastatic tumor cells behave and chemotax toward the artificial blood vessel device as they do around natural blood vessels in the primary tumor. Building on this, the cell trap of the present invention has been constructed, into which invasive cancer cells are attracted into a nano-component based collection system, their presence detected, and their cell type and expression profile determined. This is accomplished by fabricating a micro-sized, channel-shaped conduit in which a chemoattractant timed release hydrogel, electrode arrays, cell adhesives and fluidic control components are configured to create the device capable of collecting the invasive subpopulation of tumor cells in primary tumors.

The present invention, with its associated assay protocols for collection and interrogation of collected cells, is uniquely configured to allow direct implantation of the cell trap of the present invention in human tumors for several days. The goal of using the device and its associated assays is to quantify the number of invasive tumor cells per tumor volume, and to profile the collected tumor cells for stem cell-like properties, and expression patterns and phenotypes known to be associated with metastasis.

The present invention allows for quantification of the number of invasive tumor cells per tumor volume. This is correlated with tumor prognosis. The collected tumor cells can be profiled for stem cell-like properties. Stem-likeness is correlated with radio- and chemo-resistance. Additionally, the collected tumor cells can be profiled for expression patterns and phenotypes known to be associated with metastasis, which is correlated with drug resistance, prognosis, and treatment options.

The cell trap of the present invention can be inserted into the primary tumor at the time of fine needle aspiration and/or core biopsy. After several hours/days it will be retrieved and the cell contents profiled for sternness, and gene expression profiles indicative of resistance to treatment and metastasis.

The present invention will allow for long-term implantation suitable for looking at long term events requiring infiltration from cells outside the tumor and angiogenesis. Additional ligands and a longer cell-collection period will allow exploration of immune cells or migrating endothelial cells. The cell trap of the present invention, the actual collection portion of the present invention is composed of a chemoattractant source (e.g., dendromer-EGF), capsule (e.g., cell trap), counter (e.g., nanochip for electrically sensing cell arrival and cell numbers), remote reporter (e.g., passive response scanner allow reading of the chip output), retraction tool (e.g., mechanical retraction protocol to pull the cell trap out of the tissue without disrupting its contents) and micro-fluidic pore (used to expel living intact cells from the cell trap with a gentle flow of buffer). This technology will integrate with expression profiling protocols designed for use with cells collected using the needle collection and cell trap. The present invention will provide unique insights into tumor metastatic potential and will generate novel data and gene expression signatures in particular cell types that can then be queried in human breast cancers and correlated to prognosis.

### REFERENCES

1. Wyckoff, J., Jones, J., Condeelis, J. and Segal, J. (2000) A critical step in metastasis: In vivo analysis of intravasation at the primary tumor. Cancer Research 60:2504-2511.
2. Condeelis, J., Wyckoff, J., Segall, J. (2000) Imaging of cancer invasion and metastasis using green fluorescent protein. European J. Cancer 36:1671-1680.
3. Wyckoff, J. Segall, J., and Condeelis, J. (2000) The collection of the motile population of cells from a living tumor. Cancer Research 60:5401-5404.
4. Condeelis, J., Wyckoff, J., Bailly, M., Pestell, R., Lawrence, D., Backer, J. and Segall, J. (2001) Lamellipodia in invasion. Seminar in Cancer Biology 11(2):119-128.
5. Wang, W., Wyckoff, J., Oleynikov, Y., Singer, R., Frohlich, V., White, J., Segall, J. E., Condeelis, J. S. (2002) Single cell behavior in metastatic primary mammary tumors correlated with gene expression patterns revealed by molecular profiling Cancer Research. 62:6278-6288.
6. Ahmed, F, Wyckoff, J, Lin, E, Wang, W, Wang, Y, Hennighausen, L, Miyazaki, J, Jones, J, Pollard J, Condeelis, J S, and Segall, J. (2002) GFP Expression in the Mammary Gland for Imaging of Mammary Tumor Cells in Transgenic Mice. Cancer Research. 62:7166-7169.
7. Wang, W., Wyckoff, J., Wang, Y., Bottinger, E. P., Segall, J. E., and Condeelis, J. S. (2003) Gene expression analysis on small numbers of invasive cells collected by chemotaxis from primary mammary tumors of the mouse. BMC Biotechnology. 3:13-25.
8. Condeelis, J. and Segall, J. (2003) Intravital imaging of cell movement in tumours. Nature Reviews Cancer. 3:921-931.
9. Wyckoff, J., Wang, W., Lin, E., Wang, Y., Pixley, F., Graf, T., Segall, J., Pollard, J., and Condeelis, J. S. (2004) A paracrine loop between tumor cells and macrophages is required for tumor cell migration in mammary tumors. Cancer Research 64:7022-7029.
10. Wyckoff, J., Segall, J. and Condeelis, J (2004) “Chapter 22: Single cell imaging in animal tumors in vivo” in Live Cell Imaging: a Laboratory Manual, Eds.: David L. Spector and Robert D. Goldman, Cold Spring Harbor Laboratory Press, Cold Spring Harbor, N.Y.
11. Goswami, S., Sahai, E., Wyckoff, J., Cox, D., Pixley, F., Stanley, E. R., Segall, J. and Condeelis, J. (2005) Macrophages promote the invasion of carcinoma cells in vitro via a paracrine loop. Cancer Research 65:5278-5283.
12. Wang, W., Goswami, S., Lapidus, K., Wyckoff, J., Singer, R. H., Segall, J. E., and Condeelis, J. S. (2004) Identification and testing of a gene expression signature of invasive carcinoma cells within primary mammary tumors. Cancer Research. 64:8585-8594.
13. Goswami, S., Wang, W., Wyckoff, J. and Condeelis, J (2004) Breast Cancer Cells Isolated by Chemotaxis from Primary Tumors Show Increased Survival and Resistance To Chemotherapy. Cancer Research 64:7664-7667.
14. Wyckoff, J., Wang, Y., Lin, E., Li, J., Stanley, E. R., Segall, J., Pollard, J., and Condeelis, J. (2006) Direct visualization of macrophage assisted tumor cell motility and intravasation in mammary tumors. Cancer Cell In review.
15. Wang, Goswami, Sahai, Wyckoff, Segall, Condeelis (2005) Tumor Cells caught in the act of invading: How they revealed their strategy for enhanced cell motility. Trends Cell Biology 15:138-145.
16. Condeelis and Pollard (2006) Macrophages: Obligate partners for tumor cell migration, invasion, and metastasis. Cell 124: 263-266.
17. Weigang Wang, Ghassan Mouneimne, Jeffrey Wyckoff, Xiaoming Chen, Mazen Sidani, and John Condeelis. (2005) The phosphorylation status of cofilin is directly related to invasion, intravasation and metastasis of mammary tumors. J. Cell Biol. In press.
18. X. Feng, N. Tokronova, B. Xu, B. G. Szaro and J. Castracane, “Study of Cell Secretion Using MEMS-Based Arrays”, Micromachining and Microfabrication, Vol. 5345, (2004).
19. P. Chen, B. Xu, N. Tokranova, X. Feng, J. Castracane, and K. D. Gillis, “Amperometric Detection of Quantal Catecholamine Release on Micromachined Silicon Chips”, Analytical Chemistry, vol. 75, 3, (2003).
20. Yuan, H. T., C. Suri, D. N. Landon, G. D. Yancopoulos, and A. S. Woolf, Angiopoietin-2 is a site-specific factor in differentiation of mouse renal vasculature. J Am Soc Nephrol, 2000. 11(6): p. 1055-66.
21. Bankert, R. B., N. K. Egilmez, and S. D. Hess, Human-SCUD mouse chimeric models for the evaluation of anti-cancer therapies. Trends Immunol, 2001. 22(7): p. 386-93.
22. Williams, S. S., F. A. Chen, H. Kida, S. Yokata, K. Miya, M. Kato, M. P. Barcos, H. Q. Wang, T. Alosco, T. Umemoto, B. A. Croy, E. A. Repasky, and R. B. Bankert, Engraftment of human tumor-infiltrating lymphocytes and the production of anti-tumor antibodies in SCID mice. J Immunol, 1996. 156(5): p. 1908-15.
23. Shultz, L. D., P. A. Lang, S. W. Christianson, B. Gott, B. Lyons, S. Umeda, E. Leiter, R. Hesselton, E. J. Wagar, J. H. Leif, O. Kollet, T. Lapidot, and D. L. Greiner, NOD/LtSz-Rag1null mice: an immunodeficient and radioresistant model for engraftment of human hematolymphoid cells, HIV infection, and adoptive transfer of NOD mouse diabetogenic T cells. J Immunol, 2000. 164(5): p. 2496-507.
24. Uckun, F. M., H. N. Sather, B. J. Waurzyniak, M. G. Sensel, L. Chelstrom, O. Ek, M. B. Sarquis, J. Nachman, B. Bostrom, G. H. Reaman, and P. S. Gaynon, Prognostic significance of B-lineage leukemic cell growth in SCUD mice: a Children's Cancer Group Study. Leuk Lymphoma, 1998. 30(5-6): p. 503-14.
25. Angevin, E., L. Glukhova, C. Pavon, A. Chassevent, M. J. Terrier-Lacombe, A. F. Goguel, J. Bougaran, P. Ardouin, B. H. Court, J. L. Perrin, G. Vallancien, F. Triebel, and B. Escudier, Human renal cell carcinoma xenografts in SCID mice: tumorigenicity correlates with a poor clinical prognosis. Lab Invest, 1999. 79(7): p. 879-88.
26. Giovanella, B. C., D. M. Vardeman, L. J. Williams, D. J. Taylor, P. D. de Ipolyi, P. J. Greeff, J. S. Stehlin, A. Ullrich, R. Cailleau, D. J. Slamon, and et al., Heterotransplantation of human breast carcinomas in nude mice. Correlation between successful heterotransplants, poor prognosis and amplification of the HER-2/neu oncogene. Int J Cancer, 1991. 47(1): p. 66-71.
27. Sakakibara, T., Y. Xu, H. L. Bumpers, F. A. Chen, R. B. Bankert, M. A. Arredondo, S. B. Edge, and E. A. Repasky, Growth and Metastasis of Surgical Specimens of Human Breast Carcinomas in SCID Mice. Cancer J Sci Am, 1996. 2(5): p. 291.
28. Vassal, G., M. J. Terrier-Lacombe, A. Lellouch-Tubiana, C. A. Valery, C. Sainte-Rose, J. Morizet, P. Ardouin, G. Riou, C. Kalifa, and A. Gouyette, Tumorigenicity of cerebellar primitive neuro-ectodermal tumors in athymic mice correlates with poor prognosis in children. Int J Cancer, 1996. 69(2): p. 146-51.
29. Zatterstrom, U. K., Braakhuis, B. J., Wennenberg, J., Van Dongen, G. A., Attewell, R., Nauta, J. J., Laarman, D. A., and Ask, A., Growth of xenografted squamous cell carcinma of the head and neck—possible correlation with patient survival. Acta Pathol. Microbiol. Immunol. Scand., 1992. 100: p. 976-980.
30. Jessup, J. M., R. Giavazzi, D. Campbell, K. R. Cleary, K. Morikawa, R. Hostetter, E. N. Atkinson, and I. J. Fidler, Metastatic potential of human colorectal carcinomas implanted into nude mice: prediction of clinical outcome in patients operated upon for cure. Cancer Res, 1989. 49(24 Pt 1): p. 6906-10.
31. Kleine, W., Prognostic significance of growth characteristics of xenotransplanted ovarian carcinomas into nude mice. Gynecol Oncol, 1986. 25(1): p. 65-72.
32. Sasaki, T., T. Yagi, S. Ishii, T. Matsuyama, S. Yamawaki, and K. Isu, Transplantation of human osteosarcoma into nude mouse—correlation between experiment results and prognosis of patients. Nippon Seikeigeka Gakkai Zasshi, 1986. 60(2): p. 251-61.
33. Volm, M. and J. Mattern, Clinical prognostic relevance of growth of human adenocarcinomas of the lung in nude mice. Anticancer Res, 1987. 7(6): p. 1129-32.
34. Perez-Soler, R., B. Kemp, Q. P. Wu, L. Mao, J. Gomez, A. Zeleniuch-Jacquotte, H. Yee, J. S. Lee, J. Jagirdar, and Y. H. Ling, Response and determinants of sensitivity to paclitaxel in human non-small cell lung cancer tumors heterotransplanted in nude mice. Clin Cancer Res, 2000. 6(12): p. 4932-8.
35. AJCC, Cancer Staging Handbook. from the AJCC Cancer Staging Manual. 6th Edition. 2002: Springer-Verlag, Inc. 255-283.
36. Lin, E. Y., J. G. Jones, P. Li, L. Zhu, K. D. Whitney, W. J. Muller, and J. W. Pollard, Progression to malignancy in the polyoma middle T oncoprotein mouse breast cancer model provides a reliable model for human diseases. Am J Pathol, 2003. 163(5): p. 2113-26.
37. Kempiak, S. J., S. C. Yip, J. M. Backer, and J. E. Segall, Local signaling by the EGF receptor. J Cell Biol, 2003. 162(5): p. 781-7.
38. Wells, A., Tumor invasion: role of growth factor-induced cell motility. Adv Cancer Res, 2000. 78: p. 31-101.
39. Segall, J. E., S. Tyerech, L. Boselli, S. Masseling, J. Helft, A. Chan, J. Jones, and J. Condeelis, EGF stimulates lamellipod extension in metastatic mammary adenocarcinoma cells by an actin-dependent mechanism. Clin Exp Metastasis, 1996. 14(1): p. 61-72.
40. Bailly, M., L. Yan, G. M. Whitesides, J. S. Condeelis, and J. E. Segall, Regulation of protrusion shape and adhesion to the substratum during chemotactic responses of mammalian carcinoma cells. Exp Cell Res, 1998. 241(2): p. 285-99.
41. Webb, S. E., J. W. Pollard, and G. E. Jones, Direct observation and quantification of macrophage chemoattraction to the growth factor CSF-1. J Cell Sci, 1996. 109 (Pt 4): p. 793-803.
42. Ritch, P. A., S. L. Carroll, and H. Sontheimer, Neuregulin-1 enhances motility and migration of human astrocytic glioma cells. J Biol Chem, 2003. 278(23): p. 20971-8.
43. Hazar, B., S. Paydas, S. Zorludemir, B. Sahin, and I. Tuncer, Prognostic significance of microvessel density and vascular endothelial growth factor (VEGF) expression in non-Hodgkin's lymphoma. Leuk Lymphoma, 2003. 44(12): p. 2089-93.
44. Price, D. J., T. Miralem, S. Jiang, R. Steinberg, and H. Avraham, Role of vascular endothelial growth factor in the stimulation of cellular invasion and signaling of breast cancer cells. Cell Growth Differ, 2001. 12(3): p. 129-35.
45. Forough, R., L. Lindner, C. Partridge, B. Jones, G. Guy, and G. Clark, Elevated 80K-H protein in breast cancer: a role for FGF-1 stimulation of 80K-H. Int J Biol Markers, 2003. 18(2): p. 89-98.
46. George, D., Platelet-derived growth factor receptors: a therapeutic target in solid tumors. Semin Oncol, 2001. 28(5 Suppl 17): p. 27-33.
47. Krettek, A., G. Ostergren-Lunden, G. Fager, C. Rosmond, G. Bondjers, and F. Lustig, Expression of PDGF receptors and ligand-induced migration of partially differentiated human monocyte-derived macrophages. Influence of IFN-gamma and TGF-beta. Atherosclerosis, 2001. 156(2): p. 267-75.
48. Ross, R., E. W. Raines, and D. F. Bowen-Pope, The biology of platelet-derived growth factor. Cell, 1986. 46(2): p. 155-69.
49. Savikko, J. and E. von Willebrand, Coexpression of platelet-derived growth factors AA and BB and their receptors during monocytic differentiation. Transplant Proc, 2001. 33(3): p. 2307-8.
50. Parr, C., G. Watkins, R. E. Mansel, and W. G. Jiang, The hepatocyte growth factor regulatory factors in human breast cancer. Clin Cancer Res, 2004. 10(1 Pt 1): p. 202-11.
51. Burger, M., A. Glodek, T. Hartmann, A. Schmitt-Graff, L. E. Silberstein, N. Fujii, T. J. Kipps, and J. A. Burger, Functional expression of CXCR4 (CD 184) on small-cell lung cancer cells mediates migration, integrin activation, and adhesion to stromal cells. Oncogene, 2003. 22(50): p. 8093-101.
52. Muller, A., B. Homey, H. Soto, N. Ge, D. Catron, M. E. Buchanan, T. McClanahan, E. Murphy, W. Yuan, S. N. Wagner, J. L. Barrera, A. Mohar, E. Verastegui, and A. Zlotnik, Involvement of chemokine receptors in breast cancer metastasis. Nature, 2001. 410(6824): p. 50-6.
53. Mills, G. B. and W. H. Moolenaar, The emerging role of lysophosphatidic acid in cancer. Nat Rev Cancer, 2003. 3(8): p. 582-91.
54. Grimshaw, M. J., T. Hagemann, A. Ayhan, C. E. Gillett, C. Binder, and F. R. Balkwill, A role for endothelin-2 and its receptors in breast tumor cell invasion. Cancer Res, 2004. 64(7): p. 2461-8.
55. Fidler, U., Critical determinants of cancer metastasis: rationale for therapy. Cancer Chemother Pharmacol, 1999. 43(Suppl): p. S3-10.
56. Price, J. E., The biology of cancer metastasis. Prog Clin Biol Res, 1990. 354A: p. 237-55.
57. Morris, V. L., E. E. Schmidt, I. C. MacDonald, A. C. Groom, and A. F. Chambers, Sequential steps in hematogenous metastasis of cancer cells studied by in vivo videomicroscopy. Invasion Metastasis, 1997. 17(6): p. 281-96.
58. Naumov, G. N., S. M. Wilson, I. C. MacDonald, E. E. Schmidt, V. L. Morris, A. C. Groom, R. M. Hoffman, and A. F. Chambers, Cellular expression of green fluorescent protein, coupled with high-resolution in vivo videomicroscopy, to monitor steps in tumor metastasis. J Cell Sci, 1999. 112(Pt 2): p. 1835-42.
59. Kerbel, R. S., What is the optimal rodent model for anti-tumor drug testing? Cancer Metastasis Rev, 1998. 17(3): p. 301-4.
60. Killion, J. J., R. Radinsky, and I. J. Fidler, Orthotopic models are necessary to predict therapy of transplantable tumors in mice. Cancer Metastasis Rev, 1998. 17(3): p. 279-84.
61. Price, J. E., Analyzing the metastatic phemotype. J Cell Biochem, 1994. 56(1). 16-22.
62. Weiss, L., E. Mayhew, D. G. Rapp, and J. C. Holmes, Metastatic inefficiency in mice bearing B16 melanomas. Br J Cancer, 1982. 45(1): p. 44-53.
63. Butler, T. P. and P. M. Gullino, Quantitation of cell shedding into efferent blood of mammary adenocarcinoma. Cancer ERs, 1975. 35(3): p. 512-6.
64. Glaves, D., Detection of circulating metastatic cells. Prog Clin Biol Res, 1986. 212: p. 151-67.
65. Liotta, L. A., J. Kleinerman, and G. M. Saidel, Quantitative relationships of intravascular tumor cells, tumor vessels, and pulmonary metastases following tumor implantation. Cancer Res, 1974. 34(5): p. 997-1004.
66. Elston, C. W. and I. O. Ellis, Pathological prognostic factors in breast cancer. I. The value of histological grade in breast cancer” experience from a large study with long-term follow-up. Histopathology, 1991. 19(5): p. 403-10.
67. Carter, D., Interpretation of Breast Biopsies, in Rosen's Breast Pathology 2001, L. W. Wilkins, Editor. 2001, Lippincott Williams & Wilkins: New York.
68. Ramaswamy, S., K. N. Ross, E. S. Lander, and T. R. Golub, A molecular signature of metastasis in primary solid tumors. Nat Genet, 2003. 33(1): p. 49-54.
69. Bernards, R. and R. A. Weinberg, A progression puzzle. Nature, 2002. 418(6900): p. 823.
70. Szaro et. al, U.S. patent application Ser. No. 10/945,563, “Novel Biosensor Using Living Cells on Silicon-Based Microarrays,” filed Sep. 20, 2004.
71. Raja, W. et. al, “A New Chemotactic Chamber for Cell Migration Studies,” 2010.

1. A method of isolating motile cells from an animal tissue, the method comprising
implanting in the animal tissue a cell trap comprising at least one chamber with an inlet for ingress of motile cells, and a porous matrix located in the chamber comprising a chemotactic factor, for a time sufficient for the motile cells to migrate into the cell trap;
removing the implanted cell trap; and
retrieving the motile cells from the cell trap.

2. A method of isolating motile cells from an animal tissue, the method comprising
implanting in the animal tissue a cell trap comprising at least one chamber with an inlet for ingress of motile cells, an electrode array located in the chamber to indicate the presence of motile cells in the cell trap, a transmitter attached to the electrode array to transmit the indication of the presence of motile cells in the cell trap to an external data acquisition system, and a porous matrix located in the chamber comprising a chemotactic factor, for a time sufficient for the motile cells to migrate into the cell trap;
removing the implanted cell trap; and
retrieving the motile cells from the cell trap.

3. The method of claim 2, wherein the cell trap additionally comprises an etched channel extending from the inlet through the cell trap until substantially the rear of the cell trap to facilitate movement of the motile cells towards the rear of the cell trap.

4. The method of claim 2, wherein the cell trap additionally comprises an openable outlet located substantially opposite the inlet.

5. The method of claim 4, wherein the outlet is covered by a membrane.

6. The method of claim 3, wherein the cell trap comprises two chambers located along the etched channel

7. The method of claim 2, wherein the electrode array comprises a plurality of interdigitating electrodes.

8. The method of claim 2, wherein the electrode array is situated between the inlet and the porous matrix.

9. The method of claim 2, wherein the transmitter is wireless.

10. The method of claim 2, wherein the transmitter transmits to an external data acquisition system.

11. The method of claim 2, wherein the transmitter is a physical connection between the electrode array and an external data acquisition system.

12. The method of claim 2, wherein the chamber is comprised of glass.

13. The method of claim 2, wherein the chamber is composed of silicon.

14. The method of claim 2, wherein the porous matrix comprises a matrigel.

15. The method of claim 2, wherein the porous matrix comprises an interpenetrating matrix of miscible polymers.

16. The method of claim 2, wherein the porous matrix is an etched substrate.

17. The method of claim 2, wherein the chemotactic factor comprises epidermal growth factor.

18. The method of claim 2, wherein the chemotactic factor comprises CSF-1.

19. The method of claim 2, wherein the tissue is cancerous.

20. The method of claim 2, wherein motile cells of interest are separated from the other motile cells isolated from an animal tissue.

21. The method of claim 20, wherein separating the motile cells of interest from the other motile cells comprises combining the motile cells with microbeads, where the microbeads comprise a binding partner to a surface marker present on the other motile cells but not the motile cells of interest and removing the microbeads, thereby isolating the motile cells of interest.

22. The method of claim 20, wherein separating the motile cells of interest from the other motile cells comprises combining the motile cells with microbeads, where the microbeads comprise a binding partner to a surface marker present on the motile cells of interest but not the other motile cells and removing the microbeads, thereby isolating the motile cells of interest.

23. The method of claim 21, wherein the binding partner is an antibody.

24. The method of claim 23, wherein the binding partner is an antibody specific for CD11b.

25. The method of claim 20, wherein the motile cells of interest are cancer cells.

26. The method of claim 25, wherein the other motile cells comprise normal stromal cells and macrophages.

27. The method of claim 20, wherein the motile cells of interest are macrophages.

28. The method of claim 20, wherein the motile cells of interest are quantified.

29. The method of claim 20, wherein mRNA or protein expression of at least one gene in the motile cells of interest is determined.

30. The method of claim 20, wherein the expression of at least one gene is compared to the expression of at least one gene in a nonmotile cancer cell or noncancerous cell from the same tissue.
